(19) United States
(12) Patent Application Publication
(10) Pub. No.: US 2004/0019068 A1
(43) Pub. Date: Jan. 29, 2004

(54) Novel remedies or preventives for angiostenosis

(76) Inventors: Mizuo Miyazaki, Kyoto (JP); Keiichi Kamoshita, Tokyo (JP); Yoshikazu Sukenaga, Tokyo (JP); Yoshikazu Suzuki, Tokyo (JP)

Correspondence Address: Kevin S Lemack, Nields & Lemack, Suite 8, 176 E Main Street, Westboro, MA, 01581, US

(21) Appl. No.: 10/399,510

(22) PCT Filed: Oct. 18, 2001
(86) PCT No.: PCT/JP01/09131

(30) Foreign Application Priority Data
Oct. 19, 2000 (JP) 2000-318832
Sep. 18, 2001 (JP) 2001-282509

(51) Int. Cl. A61K 31/513

(52) U.S. Cl. 514/269

(57) ABSTRACT

The present invention relates to a therapeutic or prophylactic agent for an angiostenosis comprising as an active ingredient a compound having a pyrimidone skeleton and having chymase inhibiting activity, for example, a compound having a structure represented by the following Formula (II)

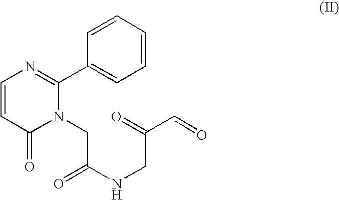

and having high selective chymase inhibiting activity or a pharmacologically acceptable salt thereof.

### TECHNICAL FIELD

The present invention relates to a therapeutic or prophylactic agent against an angiostenosis utilizing a pyrimidone skeleton compound, especially a compound having an effective in vivo inhibitory effect on a chymase activity when given orally.

### BACKGROUND ART

One of various causes of an angiostenosis is an injury in a vascular intima by some cause. At the site of such an injury, fibrin is formed, which leads to a vascular luminal stenosis due to hyperproliferation of vascular smooth muscle cells after a prolonged period, resulting in a blood flow obstruction. The cause of such hyperproliferation may for example be the development of fibrin, the induction of an inflammatory reaction, the migratory proliferation of vascular smooth muscle cells, and the like. A typical example of an angiostenosis due to a vascular injury is a restenosis developing after treatment for angina pectoris by revascularization procedures such as percutaneous transluminal coronary angioplasty (hereinafter referred to as PTCA) or coronary artery bypass graft surgery (hereinafter referred to as CABG). The restenosis after PTCA occurs in 30 to 50% of the patients receiving such treatments, posing the problems associated with this treatment. In an attempt to solve such a problem, the stent placement in the lesion, other revascularization procedures such as direct coronary atherectomy or rotablator, post-PTCA irradiation or gene therapies are applied clinically or investigated, but none of them were not successful in solving the problem. A bypass vessel jointed by the CABG is also known to undergo an injury of the bypass vessel due to pressure overload after the surgery, resulting in similar stenosis called vein graft disease. A patient having such condition can not avoid repetitive revascularization procedures.

Experiments of angiotensin converting enzyme inhibitor administrating rodents revealed that angiotensin II was an important factor in an angiostenosis (Life Sci., 54(6), PL87 (1994)). In human vessels, the production of angiotensin II is believed to involve a chymase instead of the angiotensin converting enzyme unlike to the vessels of the rodents (Japan. J. Pharmacol., 62, 207 (1993)).

Nevertheless, these understandings are only the assumptions based on in vitro experiments or indirect animal experiments, and the involvement of a chymase in an in vivo angiostenosis is rather controversial. Since there was no effective and selective inhibitor of the chymase in vivo, the role of the chymase in an angiostenosis has not been clarified yet.

Currently, a drug named tranilast was reported in clinical tests in Japan (RINSHO IYAKU, 12(1), 65 (1996)), but this drug has no chymase inhibiting effect and was revealed to cause a hepatic dysfunction as a highly frequent side effect (RINSHO IYAKU, 12(1), 65 (1996)), and so, there is a demand of a side effect-free etiotropic agent for treating or preventing a restenosis.

### DISCLOSURE OF THE INVENTION

We considered that by inhibiting a chymase selectively and suppressing the in vivo angiotensin II production in a vascular tissue a therapy or prophylaxis without side effects is possible for an angiostenosis, such as a vascular injury-induced angiostenosis, specifically a restenosis of a coronary artery resulting from a treatment of an angina pectoris by revascularization procedures, and we studied extensively using animal models and finally accomplished the invention.

Thus, the invention relates to the following (1) to (15).

(1) A therapeutic or prophylactic agent for an angiostenosis comprising as an active ingredient a compound having a pyrimidone skeleton and having a chymase inhibiting effect or a pharmacologically acceptable salt thereof;

(2) The therapeutic or prophylactic agent for an angiostenosis according to the above-mentioned (1) wherein the pyrimidone skeleton has a chemical structure represented by Formula (II):

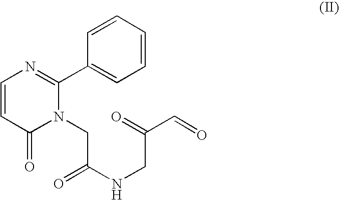

(3) A therapeutic or prophylactic agent for an angiostenosis comprising as an active ingredient a compound represented by Formula (I):

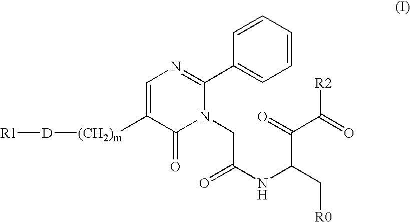

wherein R0 is an aryl group, R1 is a hydrogen atom, a C1-C6 alkyl group, a C1-C6 acyl group, a (C1-C6) alkyloxycarbonyl group optionally substituted by a monocyclic aromatic group, a (C1-C6) alkylsulfonyl group optionally substituted by an aryl group, a (C1-C6) alkylaminosulfonyl group optionally substituted by an aryl group, or a saturated heterocyclic carbonyl group, D is an oxygen atom or —NH—, m is an integer of 0 to 3, R2 is an optionally substituted (C1-C6)alkyl group, or (C1-C6)alkyloxy group or a pharmacologically acceptable salt thereof;

(4) The therapeutic or prophylactic agent for an angiostenosis according to the above-mentioned (3) wherein, in R0, the aryl group is a phenyl group optionally substituted by a (C1-C6)alkyl group or a halogen atom, in R1, the (C1-C6)alkyloxycarbonyl group optionally substituted by a monocyclic aromatic group is a (C1-C6)alkyloxycarbonyl group or a pyridyl(C1-C6)alkyloxycarbonyl group, the (C1-C6)alkylsulfonyl group optionally substituted by an aryl group is a (C1-C6)alkylsulfonyl group substituted by a phenyl group, the (C1-C6)alkylaminosulfonyl group optionally substituted by an aryl group is a (C1-C6)alkylaminosulfonyl group substituted by a phenyl group and the saturated heterocyclic carbonyl group is an oxygen-containing saturated heterocyclic carbonyl group, and in R2, the optionally substituted (C1-C6)alkyl group is a (C1-C6)alkyl group, a (C1-C6)alkyloxy(C1-C6)alkyl group, a (C1-C6)alkyl group substituted by an aryl group, a (C1-C6)alkyl group substituted by a nitrogen-containing heterocyclic oxy group or a (C1-C6)alkyl group substituted by a nitrogen-containing heterocyclic group;

(5) The therapeutic or prophylactic agent for an angiostenosis according to the above-mentioned (4) wherein the oxygen-containing saturated heterocyclic carbonyl group in the saturated heterocyclic carbonyl group in R1 is a tetrahydrofuroyl group, and, in R2, the (C1-C6)alkyl group substituted by an aryl group in the optionally substituted (C1-C6) alkyl group is a (C1-C6)alkyl group substituted by a phenyl group, the (C1-C6)alkyl group substituted by a nitrogen-containing heterocyclic oxy group is a (C1-C6)alkyl group substituted by a nitrogen-containing 6-membered heterocyclic oxy group and the (C1-C6)alkyl group substituted by a nitrogen-containing heterocyclic group is a (C1-C6)alkyl group substituted by a nitrogen-containing 6-membered heterocyclic group;

(6) The therapeutic or prophylactic agent for an angiostenosis according to the above-mentioned (3) wherein R0 is a phenyl group or a (C1-C6)alkylphenyl group, R1 is a hydrogen atom, a (C1-C6)alkyloxycarbonyl group, a (C1-C6)acyl group, a phenyl(C1-C6)alkylsulfonyl group, a pyridyl(C1-C6)alkyloxy-carbonyl group, a phenyl(C1-C6)alkylaminosulfonyl group or a (C1-C6)alkyl group, m is 0 or 1, and R2 is a pyridyloxy(C1-C6)alkyl group;

(7) The therapeutic or prophylactic agent for an angiostenosis according to the above-mentioned (3) wherein R0 is a phenyl group, R1 is a hydrogen atom, a (C1-C6)acyl group or a phenyl(C1-C6)alkylaminosulfonyl group, D is —NH—, m is 0, and R2 is a pyridyloxy(C1-C6)alkyl group;

(8) The therapeutic or prophylactic agent for an angiostenosis according to the above-mentioned (7) wherein R1 is a formyl group, acetyl group or benzylaminosulfonyl group, and R2 is a 2-pyridyloxypropyl group;

(9) The therapeutic or prophylactic agent for an angiostenosis according to the above-mentioned (3) wherein the compound represented by Formula (I) is 2-(5-acetylamino-6-oxo-2-phenyl-1,6-dihydropyrimidin-1-yl)-N-[2,3-dioxo-1-phenylmethyl-6-(2-pyridyloxy)]hexylacetamide or 2-(5-benzyl-aminosulfonylamino-6-oxo-2-phenyl-1,6-dihydropyrimidin-1-yl)-N-[2,3-dioxo-1-phenylmethyl-6-(2-pyridyloxy)]hexylacetamide or 2-(5-hydroxymethyl-6-oxo-2-phenyl-1,6-dihydropyrimidin-1-yl)—N—[2,3-dioxo-1-phenylmethyl-6-(2-pyridyloxy)]hexylacetamide;

(10) The therapeutic or prophylactic agent for an angiostenosis according to the above-mentioned (1) wherein the compound represented by Formula (I) is 2-(5-formylamino-6-oxo-2-phenyl-1,6-dihydropyrimidin-1-yl)—N—[2,3-dioxo-1-phenylmethyl-6-(2-pyridyloxy)]hexylacetamide or a pharmacologically acceptable salt thereof;

(11) The therapeutic or prophylactic agent for an angiostenosis according to the above-mentioned (1) or (3) wherein the angiostenosis is a stenosis caused by a vascular injury;

(12) The therapeutic or prophylactic agent for an angiostenosis according to the above-mentioned (1) or (3) wherein the angiostenosis is a restenosis of a coronary artery caused by revascularization procedures;

(13) An oral pharmaceutical for treating or preventing an angiostenosis comprising as an active ingredient a compound having a pyrimidone skeleton and having a chymase inhibiting activity;

(14) The oral pharmaceutical for treating or preventing an angiostenosis according to the above-mentioned (13) wherein the compound having a pyrimidone skeleton and having a chymase inhibiting activity is a compound according to the above-mentioned (3) or a pharmacologically acceptable salt thereof;

(15) An oral pharmaceutical for treating or preventing an angiostenosis comprising as an active ingredient a compound having a chymase inhibiting effect.

### BEST MODE FOR CARRYING OUT THE INVENTION

The invention is a therapeutic or prophylactic agent for an angiostenosis comprising as an active ingredient a compound having a pyrimidone skeleton and having a chymase inhibiting activity, or a pharmacologically acceptable salt thereof (hereinafter sometimes referred to as an inventive compound).

While the inventive compound is not limited particularly provided that is a compound having a pyrimidone skeleton and having a chymase inhibiting activity, it is preferably a pyrimidone skeleton-containing compound represented by Formula (II) shown above, more preferably a compound represented by Formula (I) shown above.

A chymase in the invention is classified to a chymotrypsin type protease among serine proteases, and is a cytopathic enzyme (protein) which is stored in secretory granules in a mast cell and released in response to a stimulation. The inhibitory activity on a chymase can be determined by a known method, such as the method disclosed in WO98/09949 and WO99/41277. For example, a method using a synthetic substrate (succinyl-leucyl-leucyl-valyl-tyrosyl-methylcoumarylamide) or a method using angiotensin I may be employed. The chymase inhibiting activity is regarded to be present when the IC50 value measured by the method in the publications described above is not higher than 100 nM, preferably not higher than 50 nM, especially not higher than 20 nM. Since the chymase is different between animal species, a chymase inhibiting activity in a subject animal within the range specified above is preferred. In human, human chymase inhibiting activity within the range specified above is preferred.

An angiostenosis in the invention includes all angiostenotic conditions known generally, among which a representative condition is a vascular injury-induced angiostenosis. Such a vascular injury-induced angiostenosis is a stenosis of the vascular lumen due to an excessive cell growth occurring in the process of the repair of a vascular initial injury as a result of a substantial change in a vascular tissue, fibrin development, pressure overload, excessive blood flow, chemical stimulation, mechanical stimulation and the like. More specifically, a restenosis for example of a coronary artery as a result of revascularization procedures such as a PCTA or CABG may be contemplated. Accordingly, an angiostenosis in the invention includes a vascular restenosis.

A therapeutic agent for an angiostenosis according to the invention is a drug for treating an angiostenosis by administering an effective amount of an active ingredient compound, for example, an inventive compound described above, to a warm-blooded animal (including human) having an angiostenosis described above, while a prophylactic agent is a drug for preventing an angiostenosis by administering an effective amount of an active ingredient compound, for example, an inventive compound described above, to a warm-blooded animal (including human) having a vascular injury.

While administering an inventive compound for prophylactic treatment is preferred usually in the invention, administering an inventive compound for therapeutic treatment is also possible.

Moreover, the invention is a therapeutic or prophylactic agent for an angiostenosis comprising as an active ingredient a compound represented by Formula (I) shown above or a pharmacologically acceptable salt thereof.

In Formula (I) shown above, R0 is an aryl group. The aryl group may for example be an optionally substituted phenyl group, more preferably a phenyl group optionally substituted by a (C1-C6)alkyl group or a halogen group, most preferably a phenyl group.

A (C1-C6)alkyl group in the invention, unless otherwise specified, may for example be methyl group, ethyl group, n-propyl group, i-propyl group, n-butyl group, i-butyl group, sec -butyl group, tert-butyl group, n-pentyl group, sec-pentyl group, tert-amyl group, n-hexyl group, 1,2-dimethyl-butyl group and the like, with a (C1-C4)alkyl group such as methyl group, ethyl group, n-propyl group, n-butyl group, tert-butyl group and the like being preferred.

A halogen atom may for example be a fluorine, chlorine, bromine, iodine and the like. A phenyl group substituted by a (C1-C6)alkyl group may for example be a tolyl group, a xylyl group and the like, while a phenyl group substituted by a halogen atom may for example be a fluorophenyl group.

R1 is a hydrogen atom, a C1-C6 alkyl group, a C1-C6 acyl group, a (C1-C6)alkyloxycarbonyl group optionally substituted by a monocyclic aromatic group, a (C1-C6)alkylsulfonyl group optionally substituted by an aryl group, a (C1-C6)alkyl-aminosulfonyl group optionally substituted by an aryl group or a saturated heterocyclic carbonyl group.

A C1-C6 alkyl group may for example be a group exemplified for the (C1-C6) alkyl group described above, and a C1-C4) alkyl group is preferred, and such a (C1-C4) alkyl group may for example be those exemplified above.

A C1-C6 acyl group may for example be formyl group, acetyl group, propionyl group, butyryl group, isobutyryl group, valeryl group and the like, with formyl group and acetyl group being preferred especially.

A monocyclic aromatic group as a substituent on a (C1-C6)alkyloxycarbonyl group may for example be phenyl group, pyridyl group, pyrimidyl group, pyrazyl group, pyridazyl group, furyl group, pyrrolyl group and the like, with a pyridyl group being preferred. A (C1-C6)alkyl group in a (C1-C6)alkyloxycarbonyl group may for example be a group exemplified for the (C1-C6) alkyl group described above, and a (C1-C4) alkyl group is preferred, and such a (C1-C4) alkyl group may for example be those exemplified above.

A (C1-C6)alkyloxycarbonyl group optionally substituted by a monocyclic aromatic group may for example be a methoxycarbonyl group, ethoxycarbonyl group, n-propoxycarbonyl group, n-butoxycarbonyl group, tert-butoxycarbonyl group, n-pentyloxycarbonyl group, sec-pentyloxycarbonyl group, 2,2-dimethyl-propoxycarbonyl group, n-hexyloxycarbonyl group, 1,2-dimethyl-butyloxycarbonyl group, pyridylmethoxycarbonyl group, pyridylethoxycarbonyl group, pyridylpropoxycarbonyl group, pyridylbutoxycarbonyl group, pyrimidylmethoxycarbonyl group, pyrimidylpropoxycarbonyl group, pyrazylmethoxycarbonyl group, pyrazylbutoxycarbonyl group, pyridazylmethoxycarbonyl group, furylmethoxycarbonyl group, pyrrolylethoxycarbonyl group and the like, with a (C1-C4)alkylalkyloxycarbonyl group such as a methoxycarbonyl group, ethoxycarbonyl group, tert-butoxycarbonyl group and the like, a pyridyl(C1-C4)alkyloxycarbonyl group such as pyridylmethoxycarbonyl group, being preferred.

An aryl group as a substituent on a (C1-C6)alkylsulfonyl group may for example be one similar to an aryl group in R0, with a phenyl group being preferred. A C1-C6)alkyl group in a C1-C6)alkylsulfonyl group may for example be a group exemplified for the (C1-C6) alkyl group described above, and a (C1-C4) alkyl group is preferred, and such a (C1-C4) alkyl group may for example be those exemplified above.

A (C1-C6)alkylsulfonyl group optionally substituted by an aryl group may for example be a benzylsulfonyl group, phenethylsulfonyl group, phenylbutylsulfonyl group and the like, with a benzylsulfonyl group being preferred.

An aryl group as a substituent on a (C1-C6)alkylaminosulfonyl group may for example be one similar to an aryl group in R0, with a phenyl group being preferred. A (C1-C6)alkyl group in a (C1-C6)alkylsulfonyl group may for example be a group exemplified for the (C1-C6) alkyl group described above, and a (C1-C4) alkyl group is preferred, and such a (C1-C4) alkyl group may for example be those exemplified above.

A (C1-C6)alkylaminosulfonyl group optionally substituted by an aryl group may for example be a (C1-C6) alkylaminosulfonyl group substituted by a phenyl group, with a (C1-C4)alkylaminosulfonyl group substituted by a phenyl group such as a benzylaminosulfonyl group, phenethylaminosulfonyl group, phenylpropylaminosulfonyl group and the like being preferred, and a benzylaminosulfonyl group being preferred especially.

A preferred saturated heterocyclic carbonyl group may for example be a 5- or 6-membered saturated heterocyclic carbonyl group containing 1 to 4 heteroatoms selected from the group consisting of oxygen, nitrogen and sulfur, and those which may be exemplified are a thiolylcarbonyl group, dioxanylcarbonyl group, oxothianylcarbonyl group, dithiazinylcarbonyl group, oxathiolylcarbonyl group, pyrrolidinocarbonyl group, piperidylcarbonyl group, 4-alkyl-piperazinylcarbonyl group, morpholylcarbonyl group, tetrahydrofuroyl group and the like, with an oxygen-containing saturated heterocyclic carbonyl group being preferred, with an oxygen-containing saturated 5-membered heterocyclic carbonyl group such as tetrahydrofuroyl group being more preferred, with a 3-tetrahydrofuroyl group being preferred especially.

D is an oxygen atom or —NH—, m is an integer of 0 to 3, m is preferably 0 to 2, and more preferably D is an oxygen atom and m is 1 or D is —NH— and m is 0.

R2 is an optionally substituted (C1-C6)alkyl group or a (C1-C6)alkyloxy group. The (C1-C6)alkyl group may for example be methyl group, ethyl group, n-propyl group, i-propyl group, n-butyl group, i-butyl group, sec-butyl group, tert-butyl group, n-pentyl group, sec-pentyl group, tert-amyl group, n-hexyl group, 1,2-dimethyl-butyl group and the like, with a (C1-C4)alkyl group such as methyl group, ethyl group, n-propyl group, n-butyl group, tert-butyl group and the like being preferred.

A substituted (C1-C6)alkyl group in R2 may for example be a (C1-C6)alkyloxy(C1-C6)alkyl group, a (C1-C6)alkyl group substituted by an aryl group, a (C1-C6)alkyl group substituted by a heterocyclic oxy group, a (C1-C6)alkyl group substituted by a heterocyclic group and the like.

A (C1-C6)alkyloxy group in the invention may for example be a group formed by binding an oxygen atom to an alkyl group exemplified for the (C1-C6) alkyl group described above. A preferred alkyloxy group may for example be a (C1-C4)alkyloxy group such as methoxy group, ethoxy group, n-propoxy group, isopropoxy group, n-butoxy group, tert-butoxy group and the like.

An aryl group in a (C1-C6)alkyl group substituted by an aryl group may for example be an optionally substituted phenyl group, more preferably a phenyl group. A preferred aryl (C1-C6)alkyl group may for example be benzyl group, phenethyl group, phenylpropyl group, phenylbutyl group, phenylpentyl group, phenylhexyl group and the like, with a phenyl-substituted (C1-C4)alkyl group being more preferred. Especially, a phenethyl group and a phenylpropyl group are preferred.

A heterocyclic oxy group in a (C1-C6)alkyl group substituted by a heterocyclic oxy group is preferably a heteroaryloxy group, such as a 5- or 6-membered heterocyclic oxy group containing 1 to 4 heteroatoms selected from the group consisting of oxygen, nitrogen and sulfur, for example, a pyridyloxy group, pyrimidyloxy group, pyrazyloxy group, pyridazyloxy group, furyloxy group, pyrrolyloxy group and the like, and a nitrogen-containing heterocyclic oxy group is preferred and a nitrogen-containing 6-membered heterocyclic oxy group such as a pyridyloxy group is preferred.

A (C1-C6)alkyl group substituted by a heterocyclic oxy group may for example be a heteroaryloxy(C1-C6)alkyl group such as a pyridyloxymethyl group, pyridyloxypropyl group, pyrimidyloxymethyl group, pyrimidyloxypropyl group, pyrazyloxymethyl group, pyrazyloxybutyl group, pyridazyloxyethyl group, furyloxymethyl group, pyrrolyloxyethyl group and the like, and a 5- or 6-membered heterocyclic oxy C1-C6)alkyl group is preferred, and a nitrogen-containing 6-membered heterocyclic oxy (C1-C6)alkyl group is more preferred, and a pyridyloxy(C1-C6)alkyl group such as a pyridyloxypropyl group and the like is especially preferred.

A heterocyclic group in a (C1-C6)alkyl group substituted by a heterocyclic group may for example be a morpholinyl group, oxodihydropyridinyl group, piperidinyl group, piperazinyl group, dioxanyl group and the like, and a 4-morpholinyl group and 2-oxo-1,2-dihydropyridin-1-yl group are preferred, and a 6-membered heterocyclic group having a nitrogen atom as a heteroatom, such as 2-oxo-1,2-dihydropyridin-1-yl, is more preferred.

A C1-C6)alkyl group substituted by a heterocyclic group may for example be pyridylmethyl group, pyridylpropyl group, pyrimidylmethyl group, pyrimidylpropyl group, pyrazylmethyl group, pyrazylbutyl group, pyridazylethyl group, furylmethyl group, pyrrolylethyl group, 2-oxo-1,2-dihydropyridin-1-yl-methyl group and the like, and 2-oxo-1,2-dihydropyridin-1-yl-methyl group is preferred.

A preferred combination of R0, R1, D, m and R2 is, on the basis of the potency of the activity, is exemplified as one case in which R0 is a phenyl group or a (C1-C6)alkylphenyl group, R1 is a hydrogen atom, a (C1-C4)alkyloxycarbonyl group, a (C1-C6)acyl group, a phenyl(C1-C6)alkylsulfonyl group, a pyridyl(C1-C4)alkyloxycarbonyl group, a phenyl(C1-C6)alkyl-aminosulfonyl group or a (C1-C6)alkyl group, D is an oxygen atom or —NH—, m is 0 or 1, and R2 is a pyridyloxy(C1-C6)alkyl group.

A more preferred combination of R0, R1, D, m and R2 is, on the basis of the effect in human, is exemplified as one case in which R0 is a phenyl group, R1 is a (C1-C6)acyl group or a phenyl(C1-C6)alkylaminosulfonyl group, D is —NH—, m is 0, and R2 is a pyridyloxy(C1-C6)alkyl group.

A further preferred combination of R0, R1, D, m and R2 is, on the basis of oral absorption, is exemplified as one case in which R0 is a phenyl group, R1 is formyl group, acetyl group or benzylaminosulfonyl group, D is —NH—, m is 0, and R2 is a 2-pyridyloxypropyl group.

Representative compounds of Formula (I) employed in the invention are illustrated in Table 1, which is not intended to restrict the invention. In Table 1, Ph is a phenyl group, Boc is a tert-butoxycarbonyl group, Ac is an acetyl group and Me is a methyl group.

TABLE 1

| Compound | | | | | |
|---|---|---|---|---|---|
| No. | R0 | R1 | D | m | R2 |
| 1 | Ph | t-Boc | NH | 0 | 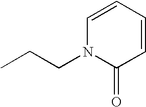 |
| 2 | Ph | H | NH | 0 | 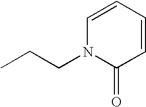 |
| 3 | Ph | t-Boc | NH | 0 | 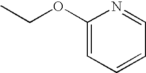 |
| 4 | Ph | H | NH | 0 | 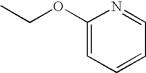 |
| 5 | Ph | t-Boc | NH | 0 | OMe |
| 6 | Ph | H | NH | 0 | OMe |
| 7 | Ph | Ac | NH | 0 | 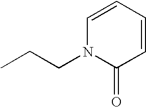 |
| 8 | Ph | 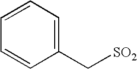 | NH | 0 | 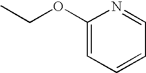 |
| 9 | Ph | 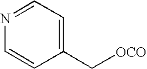 | NH | 0 | 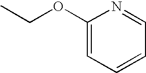 |
| 10 | Ph | HCO | NH | 0 | 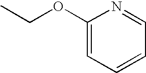 |
| 11 | Ph | Ac | NH | 0 | 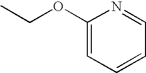 |
| 12 | Ph | 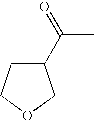 | NH | 0 | 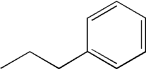 |
| 13 | 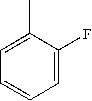 | t-Boc | NH | 0 | Me |
| 14 | Ph | H | NH | 0 | Me |
| 15 | Ph | 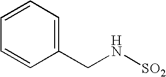 | NH | 0 | 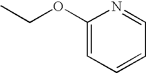 |
| 16 | Ph | Ac | O | 1 | 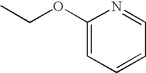 |
| 17 | Ph | Me | O | 1 | 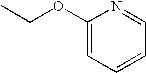 |
| 18 | Ph | H | O | 1 | 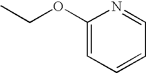 |
| 19 | 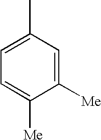 | HCO | NH | 0 | 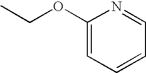 |

Among those compounds listed above, more preferred ones may for example be:

2-(5-butoxycarbonylamino-6-oxo-2-phenyl-1,6-dihydropyrimidin-1-yl)-N-[2,3-dioxo-1-phenylmethyl-6-(2-pyridyloxy)]hexylacetamide (No. 3);

2-(5-amino-6-oxo-2-phenyl-1,6-dihydropyrimidin-1-yl)-N-[2,3-dioxo-1-phenylmethyl-6-(2-pyridyloxy)]hexylacetamide (No. 4);

2-(5-benzylsulfomylamino-6-oxo-2-phenyl-1,6-dihydropyrimidin-1-yl)-N-[2,3-dioxo-1-phenylmethyl-6-(2-pyridyloxy)]hexylacetamide (No. 8);

2-(5-(4-pyridylmethoxycarbonyl)amino-6-oxo-2-phenyl-1,6-dihydropyrimidin-1-yl)-N-[2,3-dioxo-1-phenylmethyl-6-(2-pyridyloxy)]hexylacetamide (No. 9);

2-(5-formylamino-6-oxo-2-phenyl-1,6-dihydropyrimidin-1-yl)-N-[2,3-dioxo-1-phenylmethyl-6-(2-pyridyloxy)]hexylacetamide (No. 10);

2-(5-acetylamino-6-oxo-2-phenyl-1,6-dihydropyrimidin-1-yl)-N-[2,3-dioxo-1-phenylmethyl-6-(2-pyridyloxy)]hexylacetamide (No. 11);

2-(5-benzylaminosulfonylamino-6-oxo-2-phenyl-1,6-dihydropyrimidin-1-yl)-N-[2,3-dioxo-1-phenylmethyl-6-(2-pyridyloxy)]hexylacetamide (No. 15); or,

2-(5-hydroxymethyl-6-oxo-2-phenyl-1,6-dihydropyrimidin-1-yl)-N-[2,3-dioxo-1-phenylmethyl-6-(2-pyridyloxy)]hexylacetamide (No. 18);

2-(5-formylamino-6-oxo-2-phenyl-1,6-dihydropyrimidin-1-yl)-N-[2,3-dioxo-1-(3,4-dimethylphenyl)methyl-6-(2-pyridyloxy)]hexylacetamide (No. 19);

and more preferred ones are No. 10, 11, 15 and 18. No. 10 and 11 are especially preferred.

As described above, the invention is characterized by the use of a compound having a pyrimidone skeleton and having a chymase inhibiting activity, preferably a compound having a skeleton represented by Formula (II) shown above and having a chymase inhibiting activity, more preferably a compound represented by Formula (I) for treating and/or preventing an angiostenosis.

When an inventive compound is used to treat an angiostenosis, it may be administered at an effective dose to a warm-blooded animal (including human) having the angiostenosis, while when it is used to prevent an angiostenosis it may be administered at an effective dose to a warm-blooded animal (including human) having a vascular injury, especially a vascular intimal injury.

A compound employed in the invention may be a pharmacologically acceptable salt thereof, and when the compound is basic, it may be presented as a salt with a carboxylic acid, sulfonic acid, mineral acid and the like, while, when the compound is acidic, it may be presented as a salt with an alkaline metal, alkaline earth metal, organic base and the like. The carboxylic acid, sulfonic acid, mineral acid and the like may for example be acetic acid, adipic acid, benzoic acid, citric acid, fumaric acid, aspartic acid, lactic acid, malic acid, palmitic acid, salicylic acid, tartaric acid, benzenesulfonic acid, camphorsulfonic acid, toluenesulfonic acid, hydrochloric acid, hydrobromic acid, sulfuric acid, phosphoric acid and the like. The alkaline metal, alkaline earth metal, organic base and the like may for example be lithium, sodium, potassium, calcium, magnesium, barium, tetramethylammonium, tetrabutylammonium and the like.

A compound employed in the invention includes all optical isomers, racemates, diastereomers, diastereomer mixtures, individual enantiomers and enantiomer mixtures. The binding position of a substituent may be any possible position for position isomers unless otherwise specified. Various polymorphic forms of an inventive compound including a solvate such as a hydrate, a tautomer of a solvates and the like may also be employed in the invention.

A series of the compounds represented by Formula (I) in the invention may be produced by the methods disclosed in WO98/09949 and WO99/41277, which are not limitative.

The invention is also an oral pharmaceutical for treating or preventing an angiostenosis comprising as an active ingredient a compound having a pyrimidone skeleton and having a chymase inhibiting activity. Said pyrimidone skeleton is preferably a skeleton represented by Formula (II) shown above, and one especially preferred is an oral pharmaceutical for treating or preventing an angiostenosis comprising as an active ingredient a compound represented by Formula (I) shown above.

When in the invention a compound having a chymase inhibiting activity is used, it may be given alone or formulated together with an excipient or carrier into an injection formulation, tablet, granules, fine granules, powdered medicine, capsule, plaster, ointment, spray, liquid formulation, sustained release formulation and the like, which may be given orally or parenterally, or an isolated organ is immersed directly in it's solution or coated directly with it. The oral administration is preferred. An additive such as an excipient or carrier should be a pharmaceutically acceptable one, the type and composition of which may vary depending on the administration route or mode. For example an injection formulation preferably employs a sodium chloride or a saccharide such as glucose or mannitol. An oral formulation preferably employs a starch, lactose, crystalline cellulose, magnesium stearate and the like.

A pharmaceutical may be administered via an oral route or administered systemically via a parenteral route, and may also be administered by any means capable of allowing the compound to reach a lesion effectively, for example by applying directly to an affected area surface with an ointment or spray formulation, or giving directly to a lesion in the body for example via a catheter, giving as a sustained release dosage form by indwelling a stent coated with a drug, caoting directly to an isolated organ or adding in a nutrition solution in which an isolated organ is stored during an operation. An oral administration is preferred especially.

While the amount of an inventive compound in a formulation may vary depending on the formulations, it is usually 0.1 to 100% by weight, preferably 1 to 98% by weight. For example, an injection formulation usually contain 0.1 to 30% by weight, preferably 1 to 10% by weight of an active ingredient. An oral formulation is combined with additives and used in the forms of tablets, capsules, powdered medicine, granules, liquid formulation, dry syrup and the like. Capsules, tablets, granules or dust contains usually 5 to 100% by weight, preferably 15 to 99% by weight, more preferably 20 to 98% by weight of an active ingredient. The remainder consists of pharmaceutical additives.

While the dose may vary depending on a patient's age, body weight, condition of the disease and the like, it is usually 1 to 100 mg/kg day when given parenterally and 5 to 500 mg/kg day when given orally. When a solution is employed, its concentration is 10 to 1000 nM.

A compound employed in the invention has a low toxicity, and any compound undergoes a characteristically low accumulation of the toxicity when given continuously. For example, no toxicological change was observed when an inventive compound was given to a rat at a dose of 1 mg/kg once a day orally for 4 weeks, and no toxicological change was observed when 2-(5-formylamino-6-oxo-2-phenyl-1,6-Dihydropyrimidin-1-yl)-N-[2,3-dioxo-1-phenylmethyl-6-(2-pyridyloxy)]hexylacetamide (No. 10) was given to a rat at a dose of 100 mg/kg once a day orally for 2 weeks. In addition, another characteristic advantage is the possibility of giving orally as a therapeutic or prophylactic agent for an angiostenosis.

### EXAMPLES

The invention is further described in the following EXAMPLES which are not intended to restrict the invention.

### Example 1

### Restenosis Test in Dog Bypass Graft Model

(A) Bypass Graft Model Preparation

10 Male adult beagles (8 to 10 kg) purchased from NIPPON SLC were anesthetized with an intravenous administration of pentobarbital sodium at 35 mg/kg per animal. The cervical position was incised, and a right jugular vein was isolated and implanted into the carotid artery on the same side (J.Hum.Hypertens., 12 (suppl.1), S21(1999)). After implantation, the incision was sutured and the animal was raised as usual after coming out from the anesthetic.

(B) Administration of Compound

2 Groups each including 5 dogs, which had been subjected to the bypass grafting, were provided as the treatment group and the non-treatment group. In the treatment group, 2-(5-formylamino-6-oxo-2-phenyl-1,6-dihydropyrimidin-1-yl)-N-[2,3-dioxo-1-phenylmethyl-6-(2-pyridyloxy)]hexylacetamide (No. 10) was orally administered at a dose of 5 mg/kg once a day in capsules every day over a period of 33 days in total from 5 days before the bypass grafting through the day of the blood vessel sampling.

(C) Blood Vessel Sampling

28 Days after the bypass grafting, 35 mg/kg of pentobarbital sodium was given intravenously to anesthetize. The implanted vein and the jugular vein on the other side were isolated, and each was cut at the center. One of the two pieces was used to prepare a section to measure the intraluminal area, while the other was subjected to the enzyme activity assay.

(D) Pathological Analysis (Intraluminal Area Measurement, etc.)

An isolated blood vessel was fixed in a 10% formalin neutral buffer solution, embedded in a paraffin, from which a 5 μm-thick section was prepared. The section was subjected to an elastica-van Gieson staining, and examined for the area inside the intima, the area inside the media and the intraluminal area using Olympus Image Analyzer Model VM-30.

(E) Vascular Stenosis Ratio Calculation

Based on the areas obtained in (D) described above and using the following equation, a vascular stenosis ratio of each individual animal was calculated. In the equation, A is an area inside the intima, B is an intraluminal area and C is an area inside the media.

Vascular stenosis ratio (%)=[(A−B)/(C−A)]×100

(F) Chymase Activity Measurement

An isolated blood vessel was combined with a 10-fold volume (w/v) of a 20 mM sodium phosphate buffer solution (pH7.4), placed on ice, minced with scissors, and homogenized using a dounce homogenizer. After centrifuging at 20,000G for 30 minutes, a precipitate was obtained. The precipitate was combined with a 5-fold volume of 2M potassium chloride solution and 10 mM sodium phosphate buffer solution (pH7.4) containing 0.1% Nonident P-40, and homogenized. The homogenate was allowed to stand at 4° C. overnight, and then centrifuged at 20,000G for 30 minutes. The supernatant was subjected to the chymase activity measurement.

The chymase activity was measured using angiotensin I as a substrate. Thus, in a 150 mM borate buffer (pH8.5) containing 8 mM dipyridyl phosphorofluoridate, 770 μM diisopropyl phosphorofluoridate and 5 mM ethylenediamine tetraacetic acid, the reaction of 770 μM angiotensin I with a suitable amount of the centrifugation supernatant described above was conducted for 30 minutes. Immediately after completion of the reaction, angiotensin II produced was quantified using a high performance liquid chromatography, and the chymase activity of the tissue was calculated.

(G) Results

As shown in Table 2, the chymase activity in an implanted blood vessel was inhibited evidently by the administration of an inventive chymase inhibiting compound. In response to the inhibition of the chymase activity, the hypertrophy of the intima, which is observed generally in an implanted blood vessel, was inhibited. This inhibition was marked, and the intima area in the inventive compound treatment group was almost similar to that in a normal blood vessel. Based on these results, the inventive compound was hopeful sufficiently to exert the therapeutic or prophylactic effect on a vascular injury-induced angiostenosis also in a clinical practice.

TABLE 2
Implanted vascular chymase activity and stenosis degree

| | | Treatment group by |
|---|---|---|
| | Non-treatment group | Compound No. 10 |
| Chymase activity | 11.7 ± 1.88 | 4.88 ± 0.53 |
| (mU/mg protein) | | |
| intima area (mm2) | 3.12 ± 0.16 | 1.78 ± 0.12 |
| Vascular stenosis ratio (%) | 74.0 ± 4.5 | 30.9 ± 8.8 |

### Example 2

### Restenosis Test in Dog Carotid Artery Balloon Injury Model

(A) Balloon Injury Model Preparation

21 Male adult beagles (9.4 to 11.4 kg) purchased from NIPPON NOSAN KOGYO were anesthetized with an intravenous administration of pentobarbital sodium at 30 mg/kg per animal. After immobilizing each animal in the spine position, the cervical part was incised and the right and left carotid arteries were exposed. After intravenous administration of 1000 U of heparin sodium, the right carotid artery branch was incised and the tip of a balloon catheter (Baxter 4 Fr, 40 cm) was inserted from the carotid artery to the aortic arch. With the balloon being inflated with application of pressure, the catheter was drawn out to injure the intima of the blood vessel. This procedure was repeated 5 times, and the balloon catheter was taken out, and the incision in the blood vessel was ligated. After suturing the skin incision, 1000 U of heparin sodium and 100 mg of ampicillin sodium were administered intramuscularly for the postoperative maintenance.

(B) Administration of Compound

3 Groups each including 7 balloon-injured dogs were divided as the high dose treatment group, low dose treatment group and non-treatment group. 2-(5-Formylamino-6-oxo-2-phenyl-1,6-dihydropyrimidin-1-yl)-N-[2,3-dioxo-1-phenylmethyl-6-(2-pyridyloxy)]hexylacetamide (No. 10) was given at a dose of 1 mg/kg in the high dose treatment group and at 0.2 mg/kg in the low dose treatment group once a day in capsules every day over a period of 33 days in total from 5 days before the balloon injury through the day of the blood vessel sampling. In the non-treatment group, vacant capsules were given every day. In all groups, 50 ml or more of a tap water was given forcibly after administration.

(C) Blood Vessel Sampling

Twenty eight (28) days after the balloon injury, 30 mg/kg of pentobarbital sodium was given intravenously to anesthetize. After exsanguination, the right carotid artery as an injured position was isolated, and cut into 3 pieces. About 5 mm was cut out of each of the sections which were distal, proximal and center with regard to the heart. The left carotid artery was also isolated as a non-injury position, from the center of which about 5 mm was cut off, and each was fixed in 10% formalin neutral buffer solution.

(D) Pathological Analysis (Measurement of Intraluminal Area etc.)

Each vascular tissue embedded in a paraffin was shaved into sections, and subjected to a Hematoxylin-Eoshin staining. The intraluminal area, the area inside the intima and the area inside the media were measured using Olympus Image Analyzer Model VM-30.

(E) Vascular Stenosis Ratio Calculation

Based on the areas obtained in (D) described above and using the following equation, a vascular stenosis ratio and a vascular lumen ratio of each position were calculated, and the mean of the three positions was regarded as the value of each individual animal. In the equation, A is an area inside the intima, B is an intraluminal area and C is an area inside the media.

Vascular stenosis ratio (%)=[(A−B)/(C−A)]×100

Vascular lumen ratio (%)=B/C×100

(G) Results

Each one animal in the high dose treatment group and the non-treatment group exhibited a marked thrombus in the lesion, and was excluded from the analysis. As shown in Table 3, the injury in the non-treatment group resulted in a stenosis accompanied with a marked neointimal formation, while this intimal hypertrophy was suppressed in the treatment groups in a dose-related manner and the vascular intraluminal area was held. These effects were remarkable especially in the high dose treatment group (1 mg/kg). Based on these results, the inventive compound was hopeful sufficiently to exert a therapeutic or prophylactic effect on a diversity of the vascular injury-induced angiostenosis also in a clinical practice.

TABLE 3
Vascular stenosis ratio, vascular lumen ratio, chymase
activity and stenosis degree

| | | Treatment group | |
|---|---|---|---|
| | Non- | by Compound No. 10 | |
| | treatment group | Low dose | High dose |
| Vascular stenosis ratio (%) | 18.4 ± 1.7 | 13.6 ± 2.0 | 10.5 ± 1.8* |
| Vascular lumen ratio (%) | 32.4 ' 3.6 | 38.4 ± 2.0 | 41.6 ± 1.1* |

*Significant difference from non-treatment group (p<0.05) (Dunnett's test).

### Example 3

### Formulation Example

Fifty (50.0) mg of 2-(5-formylamino-6-oxo-2-phenyl-1,6-dihydropyrimidin-1-yl)-N-[2,3-dioxo-1-phenylmethyl-6-(2-pyridyloxy)]hexylacetamide (No. 10) was mixed with 80.0 mg of low-substituted hydroxypropyl cellulose, 57.0 mg of lactose, 2.0 mg of hydroxypropyl cellulose and 1.0 mg of magnesium stearate, and filled in a capsule No. 3 to obtain an oral capsule.

Industrial Applicability

In the invention, a therapeutic or prophylactic agent against an angiostenosis, for example, a therapeutic or prophylactic agent against a vascular injury-induced angiostenosis, especially a therapeutic or prophylactic agent against a restenosis after revascularization procedures is provided by employing a compound having a pyrimidone skeleton and having a highly selective in vivo chymase inhibiting effect, for example, a compound represented by Formula (I) or a pharmacologically acceptable salt thereof as an active ingredient and bringing the effect of compound into a living body for example via an oral administration.

1. (Cancelled)

2. (Amended) The therapeutic or prophylactic for against an angiostenosis comprising as an active ingredient a compound having the pyrimidone skeleton represented by Formula (II): 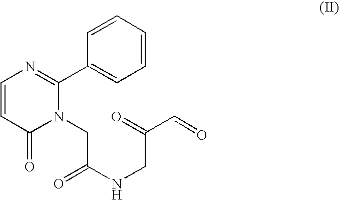
or a pharmacologically acceptable salt thereof:

11. (Amended) The therapeutic or prophylactic agent for an angiostenosis according to claim 2 or 3 wherein the angiostenosis is a stenosis caused by a vascular injury.

12. (Amended) The therapeutic or prophylactic agent for an angiostenosis according to claim 2 or 3 wherein the angiostenosis is a restenosis of a coronary artery caused by a blood flow reconstructing surgery.

13. (Amended) An oral pharmaceutical for treating or preventing an angiostenosis comprising as an active ingredient a compound having a pyrimidone skeleton represented by Formula (II) and having a chymase inhibiting activity.

14. (Amended) The oral pharmaceutical for treating or preventing an angiostenosis according to claim 13 wherein the compound having a pyrimidone skeleton represented by Formula (II) and having a chymase inhibiting activity is a compound according to claim 3 or a pharmacologically acceptable salt thereof

15. (Cancelled)

1. A therapeutic or prophylactic agent for an angiostenosis comprising as an active ingredient a compound having a pyrimidone skeleton and having a chymase inhibiting activity or a pharmacologically acceptable salt thereof.

2. The therapeutic or prophylactic agent for an angiostenosis according to claim 1 wherein the pyrimidone skeleton has a chemical structure represented by Formula (II):

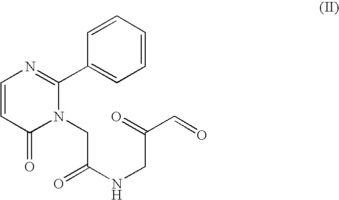

3. A therapeutic or prophylactic agent for an angiostenosis comprising as an active ingredient a compound represented by Formula (I): 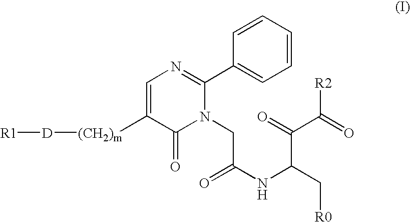
wherein R0 is an aryl group, R1 is a hydrogen atom, a C1-C6 alkyl group, a C1-C6 acyl group, a (C1-C6) alkyloxycarbonyl group optionally substituted by a monocyclic aromatic group, a (C1-C6) alkylsulfonyl group optionally substituted by an aryl group, a (C1-C6) alkylaminosulfonyl group optionally substituted by an aryl group, or saturated heterocyclic carbonyl group, D is an oxygen atom or —NH—, m is an integer of 0 to 3, R2 is an optionally substituted (C1-C6)alkyl group or (C1-C6)alkyloxy group, or a pharmacologically acceptable salt thereof.

4. The therapeutic or prophylactic agent for an angiostenosis according to claim 3 wherein, in R0, the aryl group is a phenyl group optionally substituted by a (C1-C6)alkyl group or a halogen atom, in R1, the (C1-C6)alkyloxycarbonyl group optionally substituted by a monocyclic aromatic group is a (C1-C6)alkyloxycarbonyl group or a pyridyl(C1-C6) alkyloxycarbonyl group, the (C1-C6)alkylsulfonyl group optionally substituted by an aryl group is a (C1-C6) alkylsulfonyl group substituted by a phenyl group, the (C1-C6) alkylaminosulfonyl group optionally substituted by an aryl group is a (C1-C6)alkylaminosulfonyl group substituted by a phenyl group, and the saturated heterocyclic carbonyl group is an oxygen-containing saturated heterocyclic carbonyl group, and in R2, the optionally substituted (C1-C6)alkyl group is a (C1-C6)alkyl group, a (C1-C6)alkyloxy(C1-C6)alkyl group, a (C1-C6)alkyl group substituted by an aryl group, a (C1-C6)alkyl group substituted by a nitrogen-containing heterocyclic oxy group or a (C1-C6)alkyl group substituted by a nitrogen-containing heterocyclic group.

5. The therapeutic or prophylactic agent for an angiostenosis according to claim 4 wherein the oxygen-containing saturated heterocyclic carbonyl group in the saturated heterocyclic carbonyl group in R1 is a tetrahydrofuroyl group, and, in R2, the (C1-C6)alkyl group substituted by an aryl group in the optionally substituted (C1-C6) alkyl group is a (C1-C6)alkyl group substituted by a phenyl group, the (C1-C6)alkyl group substituted by a nitrogen-containing heterocyclic oxy group is a (C1-C6)alkyl group substituted by a nitrogen-containing 6-membered heterocyclic oxy group, and the (C1-C6)alkyl group substituted by a nitrogen-containing heterocyclic group is a (C1-C6)alkyl group substituted by a nitrogen-containing 6-membered heterocyclic group.

6. The therapeutic or prophylactic agent for an angiostenosis according to claim 3 wherein R0 is a phenyl group or a (C1-C6)alkylphenyl group, R1 is a hydrogen atom, a (C1-C6)alkyloxycarbonyl group, a (C1-C6)acyl group, a phenyl(C1-C6)alkylsulfonyl group, a pyridyl(C1-C6)alkyloxycarbonyl group, a phenyl(C1-C6)alkylaminosulfonyl group or a (C1-C6)alkyl group, m is 0 or 1, and R2 is a pyridyloxy(C1-C6)alkyl group.

7. The therapeutic or prophylactic agent for an angiostenosis according to claim 3 wherein R0 is a phenyl group, R1 is a hydrogen atom, a (C1-C6)acyl group or a phenyl(C1-C6)alkylaminosulfonyl group, D is —NH—, m is 0, and R2 is a pyridyloxy(C1-C6)alkyl group.

8. The therapeutic or prophylactic agent for an angiostenosis according to claim 7 wherein R1 is a formyl group, acetyl group or benzylaminosulfonyl group, and R2 is a 2-pyridyloxypropyl group.

9. The therapeutic or prophylactic agent for an angiostenosis according to claim 3 wherein the compound represented by Formula (I) is 2-(5-acetylamino-6-oxo-2-phenyl-1,6-dihydropyrimidin-1-yl)-N-[2,3-dioxo-1-phenylmethyl-6-(2-pyridyloxy)]hexylacetamide or 2-(5-benzylamino-sulfonylamino-6-oxo-2-phenyl-1,6-dihydropyrimidin-1-yl)-N-[2,3-dioxo-1-phenylmethyl-6-(2-pyridyloxy)]hexylacetamide or 2-(5-hydroxymethyl-6-oxo-2- phenyl-1,6-dihydropyrimidin-1-yl)-N-[2,3-dioxo-1-phenylmethyl-6-(2-pyridyloxy)]hexylacetamide.

10. The therapeutic or prophylactic agent for an angiostenosis according to claim 3 wherein the compound represented by Formula (I) is 2-(5-formylamino-6-oxo-2-phenyl-1,6-dihydropyrimidin-1-yl)-N-[2,3-dioxo-1-phenylmethyl-6-(2-pyridyloxy)]hexylacetamide or a pharmacologically acceptable salt thereof.

11. The therapeutic or prophylactic agent for an angiostenosis according to claim 1 or 3 wherein the angiostenosis is a stenosis caused by a vascular injury.

12. The therapeutic or prophylactic agent for an angiostenosis according to claim 1 or 3 wherein the angiostenosis is a restenosis of a coronary artery caused by revascularization procedures.

13. An oral pharmaceutical for treating or preventing an angiostenosis comprising as an active ingredient a compound having a pyrimidone skeleton and having a chymase inhibiting activity.

14. The oral pharmaceutical for treating or preventing an angiostenosis according to claim 13 wherein the compound having a pyrimidone skeleton and having a chymase inhibiting activity is a compound according to claim 3 or a pharmacologically acceptable salt thereof.

15. An oral pharmaceutical for treating or preventing an angiostenosis comprising as an active ingredient a compound having a chymase inhibiting activity.
